(19) United States
(12) Patent Application Publication
(10) Pub. No.: US 2003/0060867 A1
(43) Pub. Date: Mar. 27, 2003

(54) Heart catheter with optimized probe

(76) Inventor: Dietmar Weber, Zell (DE)

Correspondence Address: ARENT FOX KINTNER PLOTKIN & KAHN, 1050 CONNECTICUT AVENUE, N.W., SUITE 400, WASHINGTON, DC, 20036, US

(21) Appl. No.: 10/226,213

(22) Filed: Aug. 23, 2002

(30) Foreign Application Priority Data
Aug. 24, 2001 (DE) 101 41 487.0

(51) Int. Cl. A61N 1/04

(52) U.S. Cl. 607/122

(57) ABSTRACT

The invention relates to a heart catheter, more particularly for the treatment of arrhythmogenic areas in a heart chamber, with a tubular guiding catheter, a probe for localization of the areas to be treated, comprising a probe body, and an optical fiber for irradiation of the pathological areas, seated in a receptacle. The maneuverability and stability of the probe is improved by means of a probe body having a proximal and a distal portion, the distal portion comprising a cavity that is confined by an essentially rigid wall, and having a larger cross-section than the receptacle for the fiber.

The invention relates to a heart catheter, particularly for the treatment of subendocardial arrhythmia, with a tubular guiding catheter, in which a probe for localization of pathological areas can be moved and placed perpendicularly on the cardial wall, according to claim 1.

Such heart catheters are used to reduce or interrupt the electrical conduction in pathological regions of a cardiac wall. In this effort, the heart catheter operates in a non-contact mode of laser irradiation in order to induce deep coagulation necrosis within the myocardium (cardiac wall). A characteristic advantage of this laser irradiation method is the avoidance of overheating, carbonisation and crater formation that, for instance, may occur in tissue treated by means of a radio frequency catheter.

A typical laser heart catheter comprises a probe that is positioned perpendicularly on the target tissue and that keeps an optical fiber at a given distance from the tissue surface. German patent DE3718139C1 discloses the basic principle of a heart catheter having a probe for perpendicular laser application. The disclosed probe has several sensors (electrodes) that are established in the cardiac wall and that are used to lead away electrical potentials. This electrode arrangement, however, imposes the risk of perforating or ripping the endocardium.

Another heart catheter with perpencicular probe placement is disclosed in U.S. Pat. No. 5,500,012. The probe has two rod-shaped electrodes pointing in the forward direction. These electrodes are pushed into the cardiac wall until the distal tip of an optical fiber touches the endocardium. Drawbacks of this arrangement are the small contact surface of the electrodes and the small distance between the electrodes reducing the information derived from the electrical signals. Due to the fiber touching the tissue and preventing cooling of the irradiated surface, carbonised channels are frequently induced in the cardiac wall.

DE 4205336 C1 discloses a heart catheter with a probe that uses a tent-like shield in order to keep the intracavitary blood stream out of the irradiation field A foil extending between the probe electrodes defines a relatively protected irradiation field. This arrangement, however, is relatively instable and cannot sufficiently withstand the intracardiac pressure. Additionally, manufacturing of this probe is rather complicated.

Thus, it is the object of the present invention to provide a heart catheter with a probe that can be easily manufactured and results in a significantly better stability. Moreover, the cardiac wall should not be mechanically hurt during the application.

This objective is achieved by the features defined in patent claim 1. Additional embodiments of the invention are the subject of further patent claims.

The present heart catheter comprises a tubular guiding catheter and a probe for localization of the areas to be treated which is placed perpendicularly on the target area. The probe is provided with a probe body including a receptacle for an optical fiber. The present heart catheter further comprises an operating device for advancing and withdrawing the probe in axial direction of the guiding catheter, and an optical fiber arranged in the probe body. The invention is mainly based on the idea of providing a distal portion of the probe with a cavity which is open or at least light transmissive towards the tissue and which is circumferentially surrounded by a rigid wall. The cavity preferably has a cross-section larger than the receptacle for the optical fiber. This allows for a stable placement of the probe in a defined position on the endocardium. The wall surrounding the cavity of the distal portion of the probe is made such that it can easily withstand the pressure resulting from blood and the moving cardiac walls, and that it can keep the blood out of the irradiation field in front of the distal fiber end, to allow for an undisturbed propagation of the radiated light in the cavity to the tissue. Due to its stability, the distal edge of the probe cannot be distorted and pressed into the laser beam, and laser-induced overheating of the probe is avoided.

For localization of target areas, the probe preferably comprises one or several electrodes. These could alternatively be placed at the outer guiding catheter.

The probe body is preferably made of a single piece and is characterized by a proximal and a distal potion. The proximal portion is preferably used to connect the probe with the operating device. The distal portion is preferably sized such that the divergent light beam radiated by the optical fiber can propagate conically towards the tissue if the fiber is positioned at a given distance from the tissue.

In order to avoid overheating of the endocardium during intracardiac irradiation, and to keep blood outside the cavity that is surrounding the optical fiber, the cavity is irrigated preferably with a physiological solution, and more particularly with physiological NaCl solution.

The probe body and, more particularly, the distal end of the probe body can have apertures, i.g. notches, through which the irrigation solution can leave the probe when being placed on the endocardium.

According to a preferred embodiment of the invention, the probe body comprises several, more particularly three, electrodes that are preferably mounted on the outer circumferential surface of the distal portion. The long distance between the electrodes improves the sensibility of the probe and the information derived from ECG curves recorded via the electrodes.

Additionally, the electrodes are preferably rigid, i.e. not flexible, and thus contribute to a stable positioning of the probe on the endocardium.

According to a preferred embodiment of the invention, the electrodes do not protrude, or only slightly protrude (preferably less than 0.5 mm, more particularly 0.2 mm or 0.1 mm) beyond the distal end of the probe body. This can almost completely eliminate the danger of mechanical damage to the endocardium.

The electrodes are preferably shaped like plates and are mounted at the outer wall of the distal portion. Compared to rod-shaped electrodes, a larger surface is in contact with the cardiac wall, and the risk of mechanical tissue damage is strongly reduced.

The flat electrode shape allows for a smooth advancement of the probe through introducer sheaths, guiding catheters or hemostatic valves. Additionally, the electrodes can be adapted to and mounted in special recesses on the outside surface of the distal portion. Preferably the electrodes flush with the outer surface of the probe body.

According to a preferred embodiment of the invention, the distal electrode edges can be corrugated or have a wave-like shape in order to further improve the stability of the probe on the endocardium.

The probe body preferably has one or several inlets to direct irrigation solution into the cavity.

The probe body itself is preferably made of a non-elastic, i.e. rigid, material as for instance plastic and is preferably made of one piece.

According to a preferred embodiment of the invention, the operating device is a flexible tube that is mounted at the proximal portion of the probe body. The proximal portion of the probe body is sized such that it can be introduced into and connected with the tube, particularly by glueing. The tube is also used to direct the irrigation solution to the cavity.

When attached to the probe, the outer surface of the tube preferably flushes with the probe body. Thus, the probe can be moved easily within the guiding catheter without any danger of getting stuck.

The invention is explained referring to the attached exemplary drawings showing:

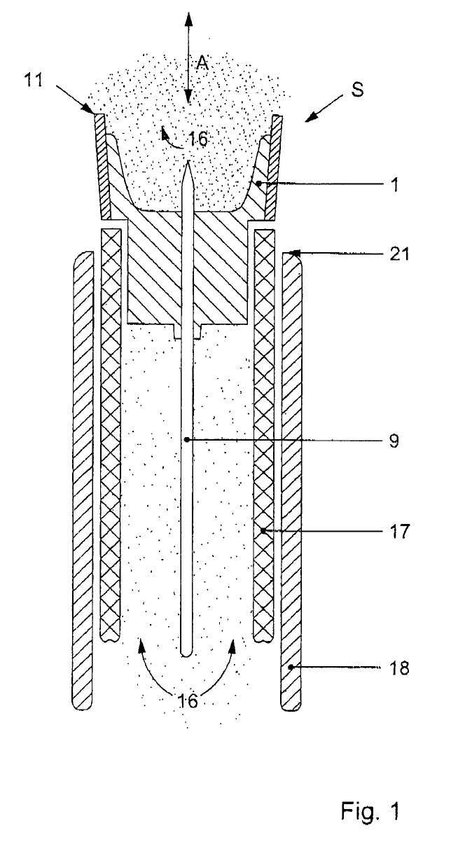

FIG. 1 a sectional view of a heart catheter with a guiding catheter and a probe movable therein

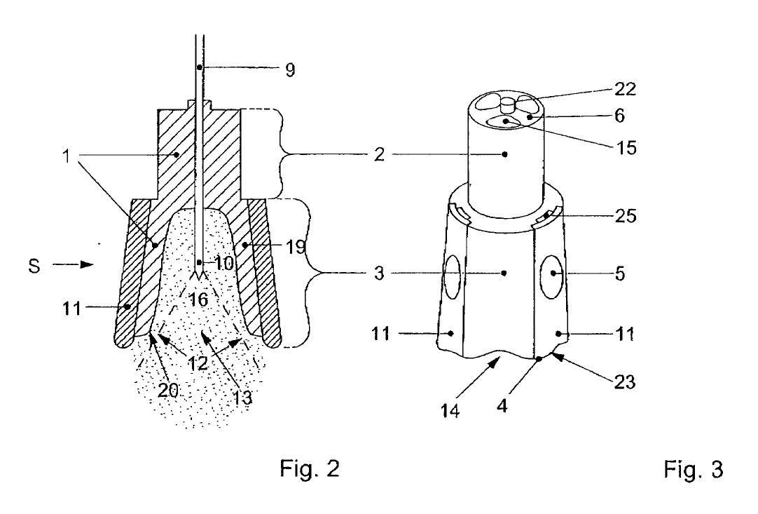

FIG. 2 a sectional view of the probe shown in FIG. 1, according to an exemplary embodiment of the invention

FIG. 3 a lateral outer view of the probe shown in FIG. 2

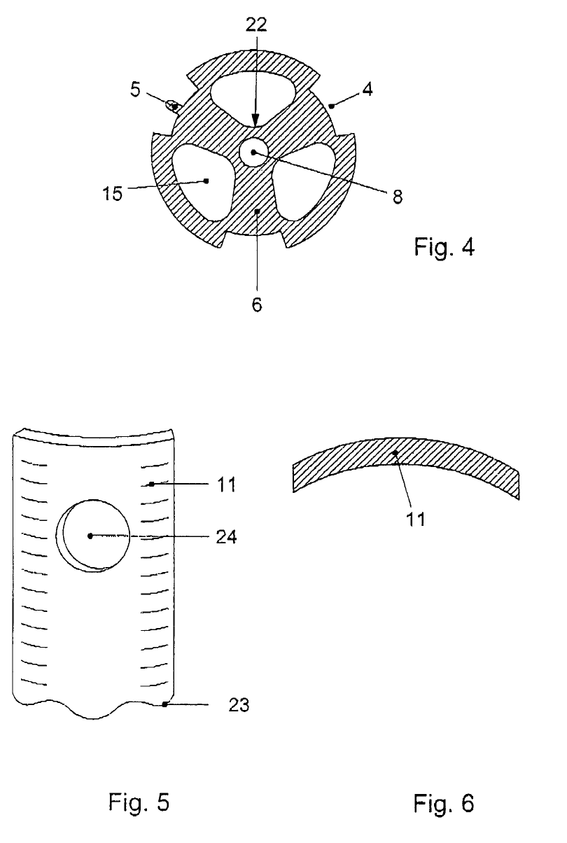

FIG. 4 a top view of the probe shown in FIG. 2

FIG. 5 a lateral view of a plate electrode; and

FIG. 6 a top view of the electrode shown in FIG. 5.

FIG. 1 shows the distal end of a heart catheter in a sectional view. The heart catheter comprises a guiding catheter 18, in which a probe S can be moved in axial direction (arrow A). The treatment of pathological areas is performed by placing the probe S in an perpendicular position on the target region, and subsequent laser irradiation of the tissue. The probe S is operated via a plastic tube 17 attached to the proximal portion 2 of the probe S.

In order to cool the endocardium and to prevent blood from entering the application cavity, physiological NaCl solution 16 is directed through the tube 17 and inlets 15 provided in a proximal portion of the probe body 1 into the application cavity 13.

The irrigation solution can leave the cavity via the notches 14 in the distal edge 20 of the probe body 1.

FIG. 2 shows an exemplary probe S in more detail. The probe S consists of the probe body 1 made of a single piece, preferably of a plastic material, with a proximal portion 2 and a distal portion 3; the proximal portion 2 having a smaller cross-section than the distal portion 3. The probe body 1 has a central receptacle 8 for receiving an optical fiber 9, the tip 10 of which protrudes into the cavity 13.

The cavity 13 (application cavity) is formed in the distal portion 3 of the probe body 1 and is surrounded by an essentially rigid wall 19. The latter is stable enough to withstand the outer blood pressure without distortion, and it can keep blood out of said cavity to allow for an undisturbed light propagation from the tip of the fiber 9 to the tissue within the cavity 13. The distal portion 3 of the probe body 1 has a distal opening at a location where the probe body 1 contacts the tissue or is at least light-transmissive in that direction.

For the treatment of pathological areas, the tip 10 of the optical fiber 9 is fixed inside the probe body 1, thereby keeping the fiber tip at a defined distance from the tissue, on which the probe is placed. If an advancement of the fiber tip 10 into the cardiac wall is desired, as it might be the case for other treatments, for example of hypertrophic myocardial diseases, the optical fiber 9 is beared inside the probe body 1 in such a way that axial movement of the fiber 9 is possible.

The cross-sectional area of the cavity 13 is significantly larger than, and in particular at least twice as large as, the cross-sectional area of the receptacle 8 for the optical fiber 9. Moreover, the axial dimension of the wall 19 is such that the light 12 radiated by the tip 10 of the optical fiber 9 can propagate freely within the cavity without hitting the distal edge 20 of the probe body 1.

The probe body 1 comprises three plate electrodes 11 that are placed in recesses 4 on the outer circumference of the distal portion 3, the outer surfaces of the electrodes 11 being flush with the outer surface of the distal portion 3. This allows for a smooth advancement of the probe through introducer sets, guiding catheters or haemostatic valves.

The tube 17, connected to the proximal portion 2, is preferably mounted flush with the outer surface of the distal portion 3.

As shown in FIG. 2, the electrodes 11 only slightly protrude beyond the edge 20 of the distal portion 3. They are particularly arranged such that they are not hit by the laser light. This prevents an excessive heating of the electrodes 11.

A lateral view of the probe body 1 in FIG. 3 clearly shows the recesses 4 and the fixing protrusions 5 provided at the outer surface of the distal portion 3 used to mount the electrodes 11 on the distal portion 3. The fixing protrusions 5 fit into corresponding holes in the electrodes 11 that are eventually fixed by a thermal treatment.

Space for connecting each electrode 11 to a wire is supplied by an additional recess 25 in the probe body 1.

The proximal portion 2 comprises several inlets 15 to direct physiological NaCl solution into the cavity 13. The inlets are separated by thin bridge walls 6. The proximal end of the probe body 1 (top) is provided with an projection 22 supporting the optical fiber 9. The projection 22 is slightly protruding beyond the proximal portion 2.

FIG. 4 shows a top view of the probe body 1 also shown in FIG. 2 with three irrigation inlets 15 which are separated by the bridge walls 6. The projection 22 with the receptacle 8 is located in the center of the probe body 1.

FIG. 5 shows a lateral view of a plate electrode 11 with its circular hole 24 fitting over the fixing protrusion 5. The plate electrode is provided with a corrugated distal edge 23 that is useful to prevent sliding of the electrode on the tissue.

FIG. 6 shows a top view of the electrode also shown in FIG. 5. The electrode 11 can be essentially characterized as a curved ring-section with a relatively large contact surface. Due to the slight protrusion of the electrodes 11 beyond the distal edge 20, and the large area that is in contact with the cardiac wall, the risk of mechanical tissue damage during the application is strongly reduced.

1. Heart catheter, particularly for the treatment of pathological areas of the cardiac walls, comprising:
a tubular guiding catheter,
a probe to be placed on a pathological tissue, having a probe body with a receptacle for an optical fiber,
an operating device for moving the probe in axial direction of the guiding catheter, and
an optical fiber having a distal tip,
characterised in that
the probe body comprises a distal portion with a cavity in which the light radiated by the tip of the optical fiber propagates to the target area, and
the cavity being circumferentially confined by an essentially rigid wall.

2. Heart catheter according to claim 1,
wherein said cavity having a cross-sectional area larger than the receptacle for the optical fiber.

3. Heart catheter according to claim 1,
wherein the cavity is sized such that a divergent light beam radiated by the optical fiber can propagate within the cavity towards the tissue, if the tip of the fiber is positioned at a given distance from the tissue.

4. Heart catheter according to claim 1,
wherein the probe body comprises a proximal portion with a small cross-section, and a distal portion with a bigger cross-section.

5. Heart catheter according to claim 1,
wherein the probe body is made of a single piece.

6. Heart catheter according to claim 1,
wherein the probe comprises one or several electrodes for localization of target areas.

7. Heart catheter according to claim 6,
wherein the electrodes are mounted on the outer circumference of the probe body

8. Heart catheter according to claim 7,
wherein the electrodes are mounted in recesses of the probe body, such that they are flush with the outer circumference of the probe body

9. Heart catheter according to claim 6,
wherein the electrodes do not or do only slightly protrude beyond a distal edge of the probe body.

10. Heart catheter according to claim 6,
wherein the electrodes have a plate-like shape.

11. Heart catheter according to claim 1,
wherein the probe body is made of a non-flexible material.

12. Heart catheter according to claim 1,
wherein the probe body comprises inlets to introduce an irrigation fluid into the cavity.
